(19) United States
(12) Patent Application Publication
(10) Pub. No.: US 2013/0110062 A1
(43) Pub. Date: May 2, 2013

(54) FEMININE SANITARY ARTICLE RETENTION AND DISPOSAL POUCH

(71) Applicant: Janette Glenn, Chicago, IL (US)

(72) Inventor: Janette Glenn, Chicago, IL (US)

(21) Appl. No.: 13/644,829

(22) Filed: Oct. 4, 2012

Related U.S. Application Data
(60) Provisional application No. 61/552,776, filed on Oct. 28, 2011.

(51) Int. Cl. A61F 13/84; A61L 15/46

(52) U.S. Cl. 604/359

(57) ABSTRACT

A feminine health pouch is provided for disposal of soiled feminine sanitary articles. The pouch includes a receptacle area for receiving the articles, an outer layer defining and surrounding a receptacle area for receiving the articles and configured for concealing the articles from public view, the receptacle area is open at one end, an inner layer adjacent to the outer layer and configured to absorb moisture and odor, and a drawstring enclosed in a channel formed from a folded over portion of the outer layer at the open end of the outer layer, the drawstring being movable for opening and closing the pouch.

This application claims priority under 35 USC 119(e) from Provisional Application Ser. No. 61/552,776 filed Oct. 28, 2011.

### FIELD OF THE INVENTION

The present invention relates generally to accessories that help women achieve modesty and discretion in matters related to personal hygiene. More specifically, this invention relates to an apparatus for modest and discreet disposal of soiled sanitary articles, such as napkins and tampons.

### BACKGROUND

Despite the increasing tolerance of publicly discussing risqué topics, the subject of menstruation is still deemed taboo. Many women and girls are embarrassed about this natural bodily function. Whether it is fear of staining one's garments, or simply being embarrassed about disposing of the tools needed to deal with it, women find the topic uncomfortable. At home, this natural monthly cycle is not so much an issue, however, when outside the home, anxiety surrounds management of this monthly occurrence. Most women can identify with the embarrassment involved in disposing of sanitary napkins and tampons during this cycle. Being discreet is of utmost importance to most people, and women prefer to take extra precautions in disposing of these napkins and tampons. Unfortunately, it is difficult to dispose of these items in a way that fully conceals or disguises them. Also, in some cases, suitable disposal is unavailable, and the soiled sanitary articles must be carried by a woman with her in her purse or elsewhere.

U.S. Pat. No. 5,579,916 is drawn to a kit for carrying feminine hygiene materials, such as individually sealed sanitary napkins. A clamshell case is provided for carrying napkins and scented disposal bags. The case includes elastic straps for retaining the contents in place, a tethered loop and a separate suction cup for suspended the case from a smooth environmental surface. Features of the case include its small size, it closes with a zipper and is bound in washable material such as vinyl or canvas.

U.S. Pat. No. 5,884,771 is drawn to a kit for use in disposing of spent medical supplies, contraceptive supplies, and feminine hygiene supplies. An outer pouch has several inner pouches, a storage pouch, and several cleaning wipes. The kit allows for the discrete storage and disposal of used supplies. Also, the outer pouch is re-sealable, opaque and colored to promote discretion. Moisture resistant and biodegradable materials are preferred for construction of the outer pouch, and a variety of plastic materials are satisfactory for construction of this pouch, which is considered disposable and inexpensive to manufacture.

There remains a need for an improved apparatus for use in disposing a soiled sanitary napkin or tampon in an inconspicuous manner.

### BRIEF DESCRIPTION OF THE DRAWINGS

FIG. 1 is a front perspective view of the present feminine health pouch; and

FIG. 2 is a partial view of the present inner layer of the pouch.

### SUMMARY

The present feminine health pouch is a clever and covert accessory that allows women to inconspicuously dispose of or temporarily store their sanitary articles. The present pouch features an opaque outer layer and an absorbent layer that, in combination, substantially reduce the emission of odor or visibility of bodily fluids once the pouch is closed. Also provided is an easy and secure closing mechanism that is preferably a drawstring.

More specifically, a feminine health pouch for disposal of soiled feminine sanitary articles is provided and includes an opaque outer layer defining and surrounding a receptacle area for receiving the articles, receptacle area is open at one end and outer layer is configured for concealing the articles, or contents of the pouch, from view. An inner layer is adjacent to the outer layer and is configured for absorbing moisture and odor, and a drawstring partially enclosed in a channel formed from a folded over portion of the outer layer is provided at the open end. The drawstring is movable for opening and closing the pouch.

### DETAILED DESCRIPTION

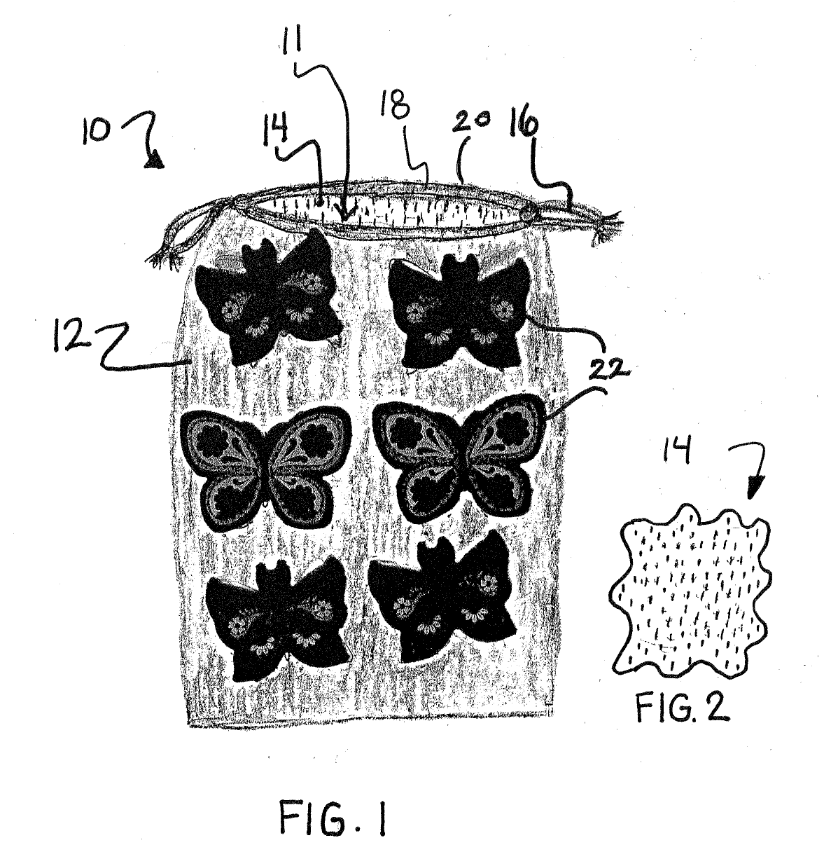

Referring now to FIG. 1, the present feminine health pouch for disposal of soiled feminine sanitary articles is generally designated 10 and includes a receptacle area 11 for receiving the articles defined and surrounded by an outer layer 12. The receptacle area 11 is open at one end and the outer layer 12 is configured to conceal the articles, or contents of the pouch 10, from public view. An inner layer 14 is adjacent to the outer layer 12 and is configured to absorb moisture and odor. A drawstring 16 is partially enclosed in a channel or sleeve 18 formed from a folded over portion of said outer layer 12 at an open end 20, the drawstring 16 being movable for opening and closing the pouch 10.

Preferably, the pouch 10 has a body that is generally rectangular in shape when viewed from the front. Other shapes, such as round and square, are contemplated. Dimensions of the pouch are preferably approximately 6 inches (15 cm) long and approximately 3 inches (7.8 cm) wide, though the pouch would be useful and can be provided in many other sizes.

The outer layer 12 is preferably made of a plastic material, and is opaque, thereby screening the contents held in the receptacle 11 of the pouch 10 from public view. More preferably, the plastic material is biodegradable. A colorful design on the outer layer may be provided to provide added discretion. Preferably, the pouch 10 is provided with the outer layer 12 in a lavender color, and is decorated with at least one design image 22, such as butterflies or the like to provide a pleasantly modest and discreet apparatus for disposing soiled sanitary articles.

Referring to FIG. 2, the absorbent inner layer 14 of the pouch 10 is preferably at least one of cotton, a cotton blend, cloth, wool and synthetic polymer material. Other absorbent materials are contemplated, as are known in the art to absorb bodily fluids as well as odors. In a preferred embodiment, the inner layer 14 is quilted, as shown in FIG. 2. It is contemplated that the inner layer 14 is secured to the outer layer 12 by stitches, adhesive, thermoforming or the like as is known in the art. Such fastening can occur at the open end 20 through stitching of the sleeve 18 for the drawstring 16, however other locations of the fastening of the layers 12, 14 are contemplated.

Use of the present pouch 10 is relatively simple. Upon the arrival of one's menstrual cycle, the user carries the pouch 10 along with their chosen pad or tampon. Once the sanitary product or article is used or soiled, the user drops it into the pouch 10, and pulls the drawstring 16 tight to retain the product in the receptacle area 11. The discarded sanitary product will then be retained and isolated from view, with any odors absorbed until the pouch 10 can be discarded in a conventional waste container.

While particular embodiments of the present apparatus for disposing of a soiled sanitary napkin have been described herein, it will be appreciated by those skilled in the art that changes and modifications may be made thereto without departing from the invention in its broader aspects and as set forth in the following claims.

What is claimed:

1. A feminine health pouch for disposal of soiled feminine sanitary articles comprising:
an outer layer defining and surrounding a receptacle area for receiving the articles and configured for concealing the articles from public view;
said receptacle area is open at one end;
an inner layer adjacent to the outer layer and configured for absorbing at least one of moisture and odor; and
a drawstring partially enclosed in a channel formed from a folded over portion of said outer layer at said open end.

2. The pouch of claim 1 having a body that is generally rectangular in shape when viewed from a front perspective view.

3. The pouch of claim 2, wherein the pouch is approximately 6 inches long and 3 inches wide.

4. The pouch of claim 1 wherein the outer layer is comprised of a plastic material.

5. The pouch of claim 4 wherein the plastic material includes a biodegradable material.

6. The pouch of claim 1 wherein the absorbent inner layer includes at least one of cotton, cotton blend, cloth, wool, and synthetic polymer materials.

7. The pouch of claim 1 wherein the absorbent inner layer is quilted.
